SEQUENCE LISTING (11 sequences)

SEQ ID NO: 1 - protein, 50 aa, Araneus diadematus
SGCDVLVQALLEVVSALVSILGSSSIGQINYGASAQYTQMVGQSVAQALA

SEQ ID NO: 2 - protein, 30 aa, Araneus diadematus
SGCDVLVQALLEVVSALVSILGSSSIGQIN

SEQ ID NO: 3 - protein, 21 aa, Araneus diadematus
SGCDVLVQALLEVVSALVSIL

SEQ ID NO: 4 - protein, 24 aa, Artificial Sequence
  His tag and start codon
MHHHHHHHHHHSSGSSLEVLFQGP

SEQ ID NO: 5 - protein, 542 aa, Artificial Sequence
  PRT204
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAA

SEQ ID NO: 6 - protein, 434 aa, Artificial
  PRT214
MHHHHHHHHHHSSGSSLEVLFQGPAGSSAAAAAAASGSGGYGPENQGPSGPVAYGPGGPVSSAAAAAAAGSGPGGYGPENQGPSGPGGYGPGGSGSSAAAAAAAASGPGGYGPGSQGPSGPGGSGGYGPGSQGASGPGGPGASAAAAAAAAAASGPGGYGPGSQGPSGPGAYGPGGPGSSAAAAAAAASGPGGYGPGSQGPSGPGVYGPGGPGSSAAAAAAAGSGPGGYGPENQGPSGPGGYGPGGSGSSAAAAAAAASGPGGYGPGSQGPSGPGGSGGYGPGSQGGSGPGASAAAAAAAASGPGGYGPGSQGPSGPGYQGPSGPGAYGPSPSASASVAASVYLRLQPRLEVSSAVSSLVSSGPTNGAAVSGALNSLVSQISASNPGLSGCDALVQALLELVSALVAILSSASIGQVNVSSVSQSTQMISQALS

SEQ ID NO: 7 - protein, 355 aa, Artificial
  PRT347
MHHHHHHSSGSSLEVLFQGPARAGPGGYRPGQQGPSGPGSAAAAAAAAAGPGGYGPGQQGPGGYGPGQQGPSGAGSAAAAAAAGPGQQGLGGYGPGQQGPGGYGPGQQGPGGYGPGSASAAAAAAGPGQQGPGGYGPGQQGPSGPGSASAAAAAAGPGGYGPGQQGPGGYAPGQQGPSGPGSAAAAAAAARAGPGGYGPAQQGPSGPGIAASAASAGPGGYGPAQQGPAGYGPGSAVAASAGAGSAGYGPGSQASAAASRLASPDSGARVASAVSNLVSSGPTSSAALSSVISNAVSQIGASNPGLSGCDVLIQALLEIVSACVTILSSSSIGQVNYGAASQFAQVVGQSVLSAF

SEQ ID NO: 8 - protein, 559 aa, Artificial
  PRT215
MHHHHHHHHHHSSGSSLEVLFQGPGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGPGGSYGPGGAGGPYGPGGPYGPGGEGPGGAGGPYGPGGVGPGGGGPGGYGPGGAGPGGYGPGGSGPGGYGPGGSGPGGYGPGGSGPGGYGPGGSGPGGYGPGGSGPGGSGPGGYGPGGSGPGGSGPGGYGPGGSGPGGYGPGGSGPGGSGPGGYGPGGSGPGGSGPGGYGPGGSGPGGFGPGGFGPGGSGPGGYGPGGSGPGGAGPGGVGPGGFGPGGAGPGGAGPGGAGPGGAGPGGAGPGGAGPGGAGPGGAGPGGAGGAGGAGGAGGSGGAGGSGGTTIIEDLDITIDGADGPITISEELTISAYYPSSRVPDMVNGIMSAMQGSGFNYQMFGNMLSQYSSGSGTCNPNNVNVLMDALLAALHCLSNHGSSSFAPSPTPAAMSAYSNSVGRMFAY

SEQ ID NO: 9 - protein, 590 aa, Artificial Sequence
  Met-PRT410
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 10 - protein, 601 aa, Artificial Sequence
  PRT410
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 11 - protein, 12 aa, Artificial Sequence
  HisTag
MHHHHHHSSGSS

(19) United States
(12) Patent Application Publication
(10) Pub. No.: US 2019/0352330 A1
(43) Pub. Date: Nov. 21, 2019

(54) Method for Recovering Protein

(71) Applicant: Spiber Inc., Yamagata (JP)

(72) Inventors: Kazuhide Sekiyama, Tsuruoka-shi, Yamagata (JP); Akihiko Ozeki, Tsuruoka-shi, Yamagata (JP); Ryoji Okada, Tsuruoka-shi, Yamagata (JP); Koichi Kotaka, Tsuruoka-shi, Yamagata (JP)

(73) Assignee: Spiber Inc., Yamagata (JP)

(21) Appl. No.: 16/473,836

(22) PCT Filed: Dec. 25, 2017
(86) PCT No.: PCT/JP2017/046394

§ 371 Date: Jun. 26, 2019

(30) Foreign Application Priority Data
Dec. 27, 2016 (JP) 2016-254005

(51) Int. Cl. C07K 1/14; C07K 14/435; C08J 11/08; C08J 11/20

(52) U.S. Cl. CPC C07K 1/145; C08J 11/20; C08J 11/08; C07K 14/43518

(57) ABSTRACT

The present invention relates to a method for recovering protein, which recovers a target protein from a mixture containing the target protein and a material different from the target protein, the method including a dissolution step of dissolving either the target protein or the material by applying pressure while heating to a solution for dissolution containing the mixture and a polar solvent, and a separation step of separating an obtained solution.

### TECHNICAL FIELD

The present invention relates to a method for recovering protein.

### BACKGROUND ART

A method of recycling (recovering) a material from waste is very useful from a point of environmental protection. Under such a circumstance, various developments have been made on a method of recycling a specific material from waste.

For example, Patent Literature 1 discloses a chemical recycling method of polyethylene terephthalate waste.

### CITATION LIST

### Patent Literature

[Patent Literature 1] Japanese Patent No. 3715812

### SUMMARY OF INVENTION

Problems to be Solved by the Invention[0005] Here, structural protein that is excellent in terms of strength and the like is a material useful as a substitute for a petroleum-derived material. However, recycling from waste is mainly related to the petroleum-derived material, and no method has been known that can recover a target protein from waste containing protein such as structural protein.

Therefore, an object of the present invention is to provide a method that can recover a target protein from a mixture containing the target protein and a material different from the target protein.

### Means for Solving the Problems

The present invention provides a method for recovering protein, which recovers a target protein from a mixture containing the target protein and a material different from the target protein, the method including a dissolution step of dissolving either the target protein or the material by applying pressure while heating to a solution for dissolution containing the mixture and a polar solvent, and a separation step of separating an obtained solution.

According to the present invention, since either the target protein or the material is dissolved by applying pressure while heating to the solution for dissolution containing the mixture and the polar solvent, for example, the target protein can be recovered from the mixture, by using solid-liquid separation.

The dissolution step is preferably a step of dissolving the target protein. Accordingly, it is possible to recover the target protein with even higher purity.

The target protein may be one or more proteins selected from the group consisting of silk fibroin, spider silk fibroin, and hornet silk fibroin.

The polar solvent preferably includes one or more solvents selected from the group consisting of water, alcohol, dimethyl sulfoxide, dimethylformamide, and hexafluoroacetone. Accordingly, it becomes easier to recover the target protein from the mixture.

The material may include one or more materials selected from the group consisting of polyester, nylon, cotton, and wool.

### Effects of the Invention

According to the present invention, it is possible to recover a target protein from a mixture containing the target protein and a material different from the target protein.

### BRIEF DESCRIPTION OF DRAWINGS

FIG. 1 is a graph showing analysis results of GPC in Example 1.

FIG. 2 is a graph showing analysis results of GPC in Example 5.

### DESCRIPTION OF EMBODIMENTS

Hereinafter, embodiments of the present invention will be described in detail. However, the present invention is not limited to the following embodiments.

A method according to the present embodiment is a method for recovering a target protein from a mixture containing the target protein and a material different from the target protein, the method including a dissolution step of dissolving either the target protein or the material by applying pressure while heating to a solution for dissolution containing the mixture and a polar solvent, and a separation step of separating an obtained solution.

When applying the pressure while heating to the solution for dissolution containing the mixture and the polar solvent, it is possible to improve solubility of either the target protein or the material different from the target protein. Accordingly, it is possible to recover the target protein from the mixture.

The dissolution step is preferably a step of dissolving the target protein. When dissolving the target protein in the solution for dissolution, it is possible to recover the target protein with higher purity.

In a case where the dissolution step is the step of dissolving the target protein, the entire amount of the target protein does not have to be dissolved, as long as a part of the protein may be dissolved.

Also, the polar solvent contained in the solution for dissolution may include, for example, one or more solvents selected from the group consisting of water, alcohol, dimethyl sulfoxide (DMSO), dimethylformamide (DMF), and hexafluoroacetone (HFA). From a viewpoint of recovering the target protein more efficiently, the polar solvent can be water alone or a mixed solvent of alcohol and water. From a viewpoint of reducing a negative impact on the environment, the polar solvent can be water. From a viewpoint of obtaining the target protein under a milder condition, the polar solvent may also contain alcohol. For example, the polar solvent may also be a mixed solvent of water and alcohol, a mixed solvent of dimethyl sulfoxide and alcohol, or a mixed solvent of water, alcohol, and dimethyl sulfoxide. A proportion of the alcohol relative to the total amount of the polar solvent (or the mixed solvent) may be 5% to 100% by mass, or 10% to 50% by mass. When the polar solvent containing alcohol is used, there is a tendency that the target protein can be dissolved in the polar solvent at a lower pressure.

In the present specification, the “alcohol” means a compound including an aliphatic group which may have a substituent and a hydroxyl group bonded to the aliphatic group. The aliphatic group may be substituted with, for example, a halogen atom such as a fluorine atom or may be unsubstituted. Examples of a fluoroalcohol having an aliphatic group substituted with a fluorine atom include hexafluoroisopropanol (HFIP).

Alcohol having a low boiling point is particularly advantageous in a point that conditions such as preparation of an alcohol solution, a concentration thereof, and formation of a formed product can be made mild. From such a viewpoint, the boiling point of the alcohol may be, for example, 99° C. or lower and 50° C. or higher at 1 atm. The boiling point of the alcohol may be 60° C. or higher at 1 atm. In addition, in general, alcohol having one hydroxyl group tends to have a lower boiling point than that of alcohol having two or more hydroxyl groups.

The carbon number of the alcohol (the carbon number of the aliphatic group) is not particularly limited, and may be 1 to 10. In particular, from the viewpoint that the target protein can be recovered under a mild condition, the carbon numbers of the alcohol may be 2 to 8 or 2 to 5. The alcohol contained in the polar solvent may be, for example, one or more alcohols having 1 to 10 carbon atoms, selected from the group consisting of methanol, ethanol, propanol, butanol, pentanol, hexanol, heptanol, octanol, nonanol, decanol, and isomers of these alcohols, one or more alcohols having 2 to 5 carbon atoms, selected from the group consisting of ethanol, propanol, butanol, and isomers of these alcohols, and one or more alcohols selected from the group consisting of ethanol, 1-propanol, and 2-propanol.

The target protein may also be a structural protein. The structural protein means protein which forms or maintains a structure, a form, and the like, in vivo. The structural protein includes hydrophobic protein and a polypeptide prone to self-aggregation in a polar solvent. Since these structural proteins generally have low solubility to a polar solvent, the method of the present embodiment is particularly useful for recovering these structural proteins.

The target protein may also be fibroin. The fibroin may be, for example, one or more selected from the group consisting of silk fibroin, spider silk fibroin, and hornet silk fibroin. The target protein may be silk fibroin, spider silk fibroin, or a combination thereof. In a case where the silk fibroin and the spider silk fibroin are used in combination, a proportion of the silk fibroin may be, for example, 40 parts by mass or less, 30 parts by mass or less, or 10 parts by mass or less, relative to 100 parts by mass of the spider silk fibroin.

Silk is a fiber obtained from a cocoon made by a silkworm that is a larva of a silkworm moth (Bombyx mori). In general, one silk thread is formed of two silk fibroin and a shell material (sericin) covering these from outside. The silk fibroin is formed of a number of fibrils. The silk fibroin is covered with four layers of the sericin. In practical, a silk filament obtained by dissolving and removing the sericin on an outer side by refining is used for a clothing application. Common silk has a specific gravity of 1.33, a fineness of 3.3 decitex on an average, and a fiber length of approximately 1300 to 1500 m. The silk fibroin is obtained by using natural or domestic silkworm cocoons or used or discarded silk fabrics, as a raw material.

The silk fibroin may be sericin-removed silk fibroin, sericin-unremoved silk fibroin, or a combination thereof. The sericin-removed silk fibroin is a powder obtained by lyophilizing silk fibroin purified by removing the sericin covering the silk fibroin and other fats and the like. The sericin-unremoved silk fibroin is unpurified fibroin from which the sericin and the like are not removed.

The spider silk fibroin may contain a spider silk polypeptide selected from the group consisting of natural spider silk protein and a polypeptide derived from the natural spider silk protein.

Examples of the natural spider silk protein include large spinneret tube dragline silk protein, weft silk protein, and minor ampullate gland protein. It is presumed that since the large spinneret tube dragline silk has a repetitive region including a crystalline region and an amorphous region, it has both high stress and stretchability. It is a major feature that the weft of spider silk has a repetitive region including an amorphous region without a crystalline region. On the other hand, the weft is inferior in stress as compared to the large spinneret tube dragline silk, but has high stretchability. It is considered that this is because most of the weft is formed of the amorphous region.

The large spinneret tube dragline silk protein has features that it is produced in a major ampullate gland of a spider and is excellent in toughness. Examples of the large spinneret tube dragline silk protein include the major ampullate spidroins MaSp1 and MaSp2 derived from an America silk spider (Nephila clavipes) and ADF3 and ADF4 derived from a European garden spider (Araneus diadematus). The ADF3 is one of two major dragline silk proteins of the European garden spider. A polypeptide derived from the natural spider silk protein may be a polypeptide derived from these dragline silk proteins. The polypeptide derived from the ADF3 is relatively easy to be synthesized, and has excellent properties in terms of strength and toughness.

The weft silk protein is produced in a flagelliform gland of the spider. Examples of the weft silk protein include flagelliform silk protein derived from the America silk spider (Nephila clavipes).

A polypeptide derived from the natural spider silk protein may be a recombinant spider silk protein. Examples of the recombinant spider silk protein include a mutant, an analogue, or a derivative of the natural spider silk protein. A preferable example of such a polypeptide is the recombinant spider silk protein of the large spinneret tube dragline silk protein (also referred to as “a polypeptide derived from the large spinneret tube dragline silk protein”).

The polypeptide derived from the large spinneret tube dragline silk protein may include two or more, five or more, or ten or more units (also referred to as a motif) of an amino acid sequence represented by Formula 1: REP1-REP2 (1). An upper limit of the number of the units of the amino acid sequence represented by Formula 1: REP1-REP2 (1) is not particularly limited, and may be, for example, 300 or less, or 200 or less. Alternatively, the polypeptide derived from the large spinneret tube dragline silk protein may be a polypeptide, having a unit of the amino acid sequence represented by Formula 1: REP1-REP2 (1), in which a C-terminus sequence is an amino acid sequence shown in any of SEQ ID NOs: 1 to 3 or an amino acid sequence having 90% or more of homology with the amino acid sequence shown in any of SEQ ID NOs: 1 to 3. In the polypeptide derived from the large spinneret tube dragline silk protein, the units of the amino acid sequence represented by Formula 1: REP1-REP2 (1) may be the same as or different from each other.

In Formula 1, a proportion of the number of alanine residues to the total number of the amino acid residues in REP1 motif is usually 83% or more, and may be 86% or more, 90% or more, or 95% or more. REP1 may be a polyalanine in which a ratio of the number of the alanine residues is 100%. Consecutive alanine (Ala) in a row in REP 1 may have two or more residues, three or more residues, four or more residues, or five or more residues. The consecutive alanine in a row in REP1 may have 20 residues or less, 16 residues or less, 12 residues or less, or 10 residues or less. REP1 motif may also include other amino acid residues selected from serine (Ser), glycine (Gly), glutamine (Gin), and the like, in addition to alanine (Ala).

In Formula 1, REP2 is an amino acid sequence consisting of 10 to 200 amino acid residues, and the total number of the residues of glycine (Gly), serine (Ser), glutamine (Gin), and alanine (Ala) contained in the amino acid sequence may be 40% or more, 60% or more, or 70% or more relative to the total number of the amino acid residues.

In the large spinneret tube dragline silk, REP1 corresponds to a crystalline region that forms a crystalline beta sheet within a fiber, and REP2 corresponds to an amorphous region that is more flexible and lacks largely regular structure. [REP1-REP2] corresponds to a repetitive region (repetitive sequence) including the crystalline region and the amorphous region, and is a characteristic sequence of a dragline silk protein.

The amino acid sequence shown in SEQ ID NO: 1 is the same as an amino acid sequence consisting of 50 amino acid residues at the C-terminus of the amino acid sequence (NCBI accession No: AAC47010, GI: 1263287) of ADF3. The amino acid sequence shown in SEQ ID NO: 2 is the same as an amino acid sequence obtained by removing 20 residues from the C-terminus of the amino acid sequence shown in SEQ ID NO: 1. The amino acid sequence shown in SEQ ID NO: 3 is the same as an amino acid sequence obtained by removing 29 residues from the C-terminus of the amino acid sequence shown in SEQ ID NO: 1.

The polypeptide including two or more units of the amino acid sequence represented by Formula 1: REP1-REP2 (1) can be, for example, a polypeptide consisting of an amino acid sequence shown in SEQ ID NO: 5. The polypeptide consisting of the amino acid sequence shown in SEQ ID NO: 5 is obtained by mutating the amino acid sequence (NCBI accession No: AAC47010, GI: 1263287) of ADF3, in which an amino acid sequence (SEQ ID NO: 4) consisting of an initiation codon, His10 tag, and Human rhinovirus 3C protease (HRV3 C protease) recognition site to an N-terminus, such that translation is terminated at a 543rd amino acid residue.

The polypeptide including two or more units of the amino acid sequence represented by Formula 1: REP1-REP2 (1) consists of an amino acid sequence in which one or more amino acids are substituted, deleted, inserted, and/or added in the amino acid sequence shown in SEQ ID NO: 5, and can be protein including a repetitive region including a crystal region and an amorphous region. In the present specification, “one or more” means, a range selected from, for example, 1 to 40, 1 to 35, 1 to 30, 1 to 25, 1 to 20, 1 to 15, 1 to 10, and 1 or several. In the present specification, “one or several” is 1 to 9, 1 to 8, 1 to 7, 1 to 6, 1 to 5, 1 to 4, 1 to 3, 1 or 2, or 1.

The polypeptide including two or more units of the amino acid sequence represented by Formula 1: REP1-REP2 (1) may be a recombinant protein derived from ADF4 having the amino acid sequence shown in SEQ ID NO: 6. The amino acid sequence shown in SEQ ID NO: 6 is obtained by adding the amino acid sequence (SEQ ID NO: 4) consisting of an initiation codon, His10 tag, and Human rhinovirus 3C protease (HRV3C protease) recognition site to the N-terminus of a partial amino acid sequence (NCBI accession No: AAC47011, GI: 1263289) of ADF4 obtained from the NCBI database.

The polypeptide including two or more units of the amino acid sequence represented by Formula 1: REP1-REP2 (1) consists of an amino acid sequence in which one or more amino acids are substituted, deleted, inserted, and/or added in the amino acid sequence shown in SEQ ID NO: 6, and can be a polypeptide including a repetitive region including a crystal region and an amorphous region.

The polypeptide including two or more units of the amino acid sequence represented by Formula 1: REP1-REP2 (1) may be a recombinant protein derived from MaSp2 having an amino acid sequence shown in SEQ ID NO: 7. The amino acid sequence shown in SEQ ID NO: 7 is obtained by adding an amino acid sequence (SEQ ID NO: 11) consisting of an initiation codon, His10 tag, and Human rhinovirus 3C protease (HRV3 C protease) recognition site to an N-terminus of a partial sequence (NCBI accession No: AAT75313, GI: 50363147) of MaSp2 obtained from the NCBI database.

The polypeptide including two or more units of the amino acid sequence represented by Formula 1: REP1-REP2 (1) consists of an amino acid sequence in which one or more amino acids are substituted, deleted, inserted, and/or added in the amino acid sequence shown in SEQ ID NO: 7, and can be a polypeptide including a repetitive region including a crystal region and an amorphous region.

The polypeptide derived from the weft silk protein may include 10 or more, 20 or more, or 30 or more units of an amino acid sequence represented by Formula 2: REP3 (2). An upper limit of the number of the units of the amino acid sequence represented by Formula 2: REP3 (2) is not particularly limited, and may be, for example, 300 or less, or 200 or less.

In Formula 2, REP3 represents an amino acid sequence consisting of Gly-Pro-Gly-Gly-X, and X represents one amino acid selected from the group consisting of alanine (Ala), serine (Ser), tyrosine (Tyr), and valine (Val).

The polypeptide including 10 or more units of the amino acid sequence represented by Formula 2: REP3 (2) may be a recombinant protein derived from flagelliform silk protein having an amino acid sequence shown in SEQ ID NO: 8. The amino acid sequence shown in SEQ ID NO: 8 is an amino acid sequence obtained in a manner that an amino acid sequence (referred to as PR1 sequence) from 1220th residue to the 1659th residue from the N-terminus, corresponding to a repeat portion and a motif of the partial sequence (NCBI accession number: AAF36090, GI: 7106224) of the flagelliform silk protein of the America silk spider obtained from the NCBI database is bonded to a C-terminus amino acid sequence from 816th residue to 907th residue from the C-terminus of the partial sequence (NCBI accession number: AAC38847, GI: 2833649) of the flagelliform silk protein of the America silk spider obtained from the NCBI database, and the amino acid sequence (SEQ ID NO: 4) consisting of an initiation codon, His10 tag, and HRV3C protease recognition site is added to an N-terminus of the bonded sequence.

The polypeptide including 10 or more units of the amino acid sequence represented by Formula 2: REP3 (2) consists of an amino acid sequence in which one or more amino acids are substituted, deleted, inserted, and/or added in the amino acid sequence shown in SEQ ID NO: 8, and can be a polypeptide including a repetitive region including an amorphous region.

From a viewpoint of productivity when performing recombinant protein production using a microorganism such as E. coli as a host, a molecular weight of the protein or the polypeptide may be 500 kDa or less, 300 kDa or less, 200 kDa or less, or 100 kDa or less, and may be 10 kDa or more.

The hornet silk fibroin is protein produced by a larva or a bee and may contain a polypeptide selected from the group consisting of natural hornet silk protein and a polypeptide derived from the natural hornet silk protein.

The polypeptide can be produced, for example, using a host transformed with an expression vector containing a gene encoding the polypeptide.

A method for producing the gene encoding the polypeptide is not particularly limited. For example, in a case of a natural spider silk protein, a gene can be manufactured by a method of amplifying and cloning the gene encoding the protein by polymerase chain reaction (PCR) or the like from cells derived from a spider, or by chemical synthesis. A chemical synthesis method of the gene is not particularly limited. For example, the gene can be chemically synthesized by a method that oligonucleotides automatically synthesized by AKTA oligopilot plus 10/100 (GE Healthcare Japan Co., Ltd.) or the like are linked by PCR and the like, based on amino acid sequence information of the natural spider silk protein obtained from the NCBI web database and the like. In this case, in order to facilitate purification and confirmation of the protein, a gene encoding protein consisting of an amino acid sequence in which an amino acid sequence including initiation codon and His10 tag is added to the N terminus of the amino acid sequence may be synthesized.

As an expression vector, a plasmid, phage, virus, or the like capable of expressing protein from a DNA sequence can be used. The plasmid expression vector is not particularly limited as long as it can express a target gene in a host cell and can amplify itself. For example, in a case where E. coli Rosetta (DE3) is used as a host, pET22b (+) plasmid vector, pCold plasmid vector, and the like can be used. Among these, from a viewpoint of productivity of protein, the pET22b (+) plasmid vector can be used. As the host, for example, an animal cell, a plant cell, and a microorganism can be used.

A combination of the target protein such as the silk fibroin and the spider silk fibroin with other proteins may be included in a solution to be obtained from the solution for dissolution and the combination. Examples of the other proteins include collagen, soy protein, casein, keratin, and whey protein. When using the other proteins in combination with the target protein, it is possible to appropriately adjust a physical property derived from the target protein. A proportion of the other proteins may be, for example, 40 parts by mass or less, 30 parts by mass or less, or 10 parts by mass or less, relative to 100 parts by mass of the target protein.

A concentration of the target protein in the solution for dissolution may be 15% by mass or more, 30% by mass or more, 40% by mass or more, or 50% by mass or more, based on the mass of the polar solvent. From a viewpoint of recovery efficiency of the target protein, the concentration of the target protein may be 70% by mass or less, 65% by mass or less, or 60% by mass or less, based on the mass of the polar solvent.

The material different from the target protein may be an inorganic material or an organic material. Examples of the inorganic material include metal, a carbon fiber, glass, or a combination thereof. Examples of the organic material include a cellulose fiber such as polyester, nylon, cotton, wool, and rayon, aramid, polytetrafluoroethylene (PTFE), polyurethane, a bioplastic fiber such as biopolyester represented by polylactic acid (PLA), bio nylon, or bioPET, or a combination thereof. Examples of the polyester include polyethylene terephthalate (PET), polyethylene naphthalate (PEN), and polytributylene terephthalate (PTT). In addition, the wool is an animal fiber of which a main component is keratin. The cotton is a vegetable fiber of which a main component is cellulose. The nylon is a hydrophobic fiber which is a type of polyamide.

The material different from the target protein may include one or more materials selected from the group consisting of polyester, nylon, cotton, and wool.

The solution for dissolution may further include one or more inorganic salts. When adding the inorganic salt to the solution for dissolution, an effect of improving the solubility by heating and pressing can be more remarkable. Examples of the inorganic salt include an inorganic salt including Lewis acid and Lewis base shown below. The Lewis base may be, for example, an oxo acid ion (such as a nitrate ion and a perchlorate ion), a metal oxo acid ion (such as a permanganate ion), a halide ion, a thiocyanate ion, and a cyanate ion. The Lewis acid may be, for example, a metal ion such as an alkali metal ion and an alkaline earth metal ion, a polyatomic ion such as an ammonium ion, and a complex ion. Specific examples of the inorganic salt include lithium salts such as lithium chloride, lithium bromide, lithium iodide, lithium nitrate, lithium perchlorate, and lithium thiocyanate, calcium salts such as calcium chloride, calcium bromide, calcium iodide, calcium nitrate, calcium perchlorate, and calcium thiocyanate, iron salts such as iron chloride, iron bromide, iron iodide, iron nitrate, iron perchlorate, and iron thiocyanate, aluminum salts such as aluminum chloride, aluminum bromide, aluminum iodide, aluminum nitrate, aluminum perchlorate, and aluminum thiocyanate, potassium salts such as potassium chloride, potassium bromide, potassium iodide, potassium nitrate, potassium perchlorate, and potassium thiocyanate, sodium salts such as sodium chloride, sodium bromide, sodium iodide, sodium nitrate, sodium perchlorate, and sodium thiocyanate, zinc salts such as zinc chloride, zinc bromide, zinc iodide, zinc nitrate, zinc perchlorate, and zinc thiocyanate, magnesium salts such as magnesium chloride, magnesium bromide, magnesium iodide, magnesium nitrate, magnesium perchlorate, and magnesium thiocyanate, barium salts such as barium chloride, barium bromide, barium iodide, barium nitrate, barium perchlorate, and barium thiocyanate, and strintium salts such as strintium chloride, strintium bromide, strintium iodide, strintium nitrate, strintium perchlorate, and strintium thiocyanate. A concentration of the inorganic salt may be 1.0% by mass or more, 5.0% by mass or more, 9.0% by mass or more, 15.0% by mass or more, or 20.0% by mass or more, based on the total mass of the target protein. The concentration of the inorganic salt may also be 30% by mass or less, 25% by mass or less, or 20% by mass or less, based on the total mass of the target protein.

The solution for dissolution can include various additives, as needed. Examples of the additive include a plasticizer, a crystal nucleating agent, an antioxidant, an ultraviolet absorber, a coloring agent, a crosslinking agent, a polymerization inhibitor, a filler, and a synthetic resin. A concentration of the additive may be 50% by mass or less, based on the total mass the target protein.

When applying a predetermined pressure while heating to the solution for dissolution, either the target protein or the material can be dissolved. Here, when applying pressure to the solution for dissolution, even at a relatively low temperature, it is possible to dissolve either the target protein or the material. Therefore, it is possible to recover the target protein while preventing denaturation, gelation, and degradation of the target protein from being caused. Further, in a case where the target protein is dissolved in the dissolution step, since a concentration step using dialysis or the like is not necessarily required, it is also possible to obtain a protein solution with high production efficiency.

The pressure applied to the solution for dissolution is adjusted such that it is possible to recover the target protein, according to types of the target protein and the polar solvent, a desired concentration, and the like. When the pressure applied to the solution for dissolution is high, the solubility tends to be higher. The pressure applied to the solution for dissolution can be 0.05 MPa or more, 0.06 MPa or more, 0.07 MPa or more, 0.08 MPa or more, 0.1 MPa or more, 1.0 MPa or more, 5.0 MPa or more, or 10 MPa or more. The pressure applied to the solution for dissolution can be 300 MPa or less, 150 MPa or less, 50 MPa or less, or 30 MPa or less.

A method of applying the pressure to the solution for dissolution is not particularly limited. For example, a method of heating a solvent and applying pressure by vapor pressure of the solvent in a pressure resistant vessel, a method of applying pressure by adjusting the pressure in a sealed pressure resistant vessel by filling the vessel with an inert gas such as nitrogen and argon or air may be applied.

The pressure may be applied to the solution for dissolution while heating the solution for dissolution. It is not limited that the heating is performed during applying the pressure. For example, the solution for dissolution is heated to a predetermined temperature and then the pressure may be applied to the solution for dissolution. From a viewpoint of further preventing the protein from degrading, a heating temperature may be 150° C. or lower, 140° C. or lower, 135° C. or lower, or 130° C. or lower. In a case where the polar solvent is water, from a viewpoint of further preventing water-mediated protein from degrading, the heating temperature is preferably 140° C. or lower. From a viewpoint of further improving the solubility, the heating temperature may be 70° C. or higher, 90° C. or higher, or 100° C. or higher. These upper and lower limits can be used in any combination. For example, the heating temperature may be 70° C. to 150° C., 90° C. to 140° C., or 100° C. to 130° C. In addition, for example, in a case where the polar solvent is a mixed solvent of water and alcohol, the heating temperature may be 150° C. or lower, 140° C. or lower, 135° C. or lower, or 130° C. or lower from the viewpoint of further preventing the protein from degrading, and may be 70° C. or higher, 80° C. or higher, or 90° C. or higher from the viewpoint of improving the solubility. These upper and lower limits can be used in any combination. For example, the heating temperature in the case where the polar solvent is the mixed solvent of water and alcohol may be 70° C. to 150° C., 80° C. to 140° C., or 90° C. to 130° C. The heating temperature may be a constant temperature or may vary.

The pressure may be applied to the solution for dissolution while stirring the solution for dissolution. It is not limited that the stirring is performed during applying the pressure. The solution for dissolution may be stirred before and after the pressure is applied thereto. A stirring method is not particularly limited. For example, the solution for dissolution can be stirred using an inclined blade, a turbine blade, or the like.

The solution obtained after pressurization may include a gas for pressurization. Therefore, the method for recovering protein according to an embodiment may further include removing a gas from the solution. A method of removing the gas is not particularly limited, and examples thereof include a method using a centrifuge. When applying the solution to the centrifuge, it is possible to remove a layer including a relatively large amount of gas.

The method for recovering protein according to the present embodiment includes the separation step of separating the solution obtained in the dissolution step.

The separation step may be performed by a usual solid-liquid separation treatment. For example, the solution and insoluble matter may be separated from each other by filtration.

In a case where the dissolution step is a step of dissolving the target protein, the concentration of the target protein dissolved in the solution is 1% by mass or more, 5% by mass or more, 10% by mass or more, 15% by mass or more, 20 mass % or more, or 30 mass % or more, and 50 mass % or less, 45 mass % or less, or 40 mass % or less, based on the mass of the solution.

The method for recovering protein according to the present embodiment may include a step of washing off insoluble matter by using a polar solvent such as water, alcohol, dimethyl sulfoxide (DMSO), dimethylformamide (DMF), hexafluoroacetone (HFA), and hexafluoro-2-propanol (HFIP). As the alcohol, the alcohols described above can be used.

In the method for recovering protein according to the present embodiment, in a case where the dissolution step is a step of dissolving the target protein, for example, the target protein can be recovered from the solution, by applying a method of removing the polar solvent from the solution and a common method such as reprecipitation. In this case, any form of protein such as powder may be recovered from the solution including the target protein, or it may be used directly to produce a formed product.

In a case where the dissolution step is a step of dissolving the target protein, the solution including the target protein can be used, for example, to produce a formed product of protein by various methods. For example, a formed product containing protein may be obtained by removing the polar solvent from the solution containing the target protein. In particular, a solution containing spider silk fibroin can be used to produce a formed product having excellent physical property making the most of the properties of the spider silk fibroin.

In a case where the dissolution step is a step of dissolving the target protein, for example, a formed product such as a gel, a film, or a fiber can be produced by using the solution as a dope solution. The film can be produced, for example, by a method of forming a film of the solution (the dope solution) and removing the polar solvent from the formed film. The fiber can be produced, for example, by a method of spinning the solution and removing the polar solvent from the spun solution.

In the method for recovering protein, it can be confirmed whether the recovered protein is the target protein, for example, using gel permeation chromatography (GPC), infrared spectroscopy (IR), polyacrylamide gel electrophoresis (SDS-PAGE), mass spectrometry (MS), and nuclear magnetic resonance (NMR).

### EXAMPLES

Hereinafter, the present invention will be described in more detail, based on Examples. However, the present invention is not limited to the following Examples.

[Preparation of Spider Silk Protein]

### (1) Synthesis of Nucleic Acid Encoding Modified Fibroin and Construction of Expression Vector

Based on a nucleotide sequence and an amino acid sequence of Nephila clavipes (GenB ank accession number: P46804.1, GI: 1174415) which is a naturally derived spider silk fibroin, a modified fibroin having the amino acid sequences respectively shown in SEQ ID NOs: 9 and 10 was designed. The amino acid sequence shown in SEQ ID NO: 9 was obtained in a manner that, starting from the naturally derived fibroin, an amino acid sequence in which the alanine residues are continuous in the (A)n motif was deleted such that the number of the consecutive alanine residues was 5, every two (A)n motif ((A)5) were deleted from an N-terminus side to a C-terminus side, and one [(A)n motif-REP] was inserted in front of the C-terminus sequence, and all GGX in REP were substituted with GQX. The amino acid sequence shown in SEQ ID NO: 10 was obtained by adding the amino acid sequence (tag sequence and hinge sequence) shown in SEQ ID NO: 11 to the N-terminus of the amino acid sequence shown in SEQ ID NO: 9.

A nucleic acid encoding protein having the amino acid sequence shown in SEQ ID NO: 10 obtained by adding the His tag sequence and the hinge sequence (SEQ ID NO: 11) to the N-terminus of the amino acid sequence shown in SEQ ID NO: 9 was synthesized. In the nucleic acid, an NdeI site was added at a 5′ end and an EcoRI site was added downstream of a stop codon. The nucleic acid was cloned into a cloning vector (pUC118). Thereafter, the same nucleic acid was cut out by restriction enzyme treatment with NdeI and EcoRI, and then recombined into a protein expression vector pET-22b (+) to obtain an expression vector.

(2) Protein Expression

E. coli BLR (DE3) was transformed with the pET 22b (+) expression vector including the nucleic acid encoding protein having the amino acid sequence shown in SEQ ID NO: 10. The transformed E. coli was cultured in 2 mL of LB medium containing ampicillin for 15 hours. The same culture solution was added to 100 mL of seed culture medium (Table 1) containing ampicillin such that an OD₆₀₀ became 0.005, to be inoculated with the transformed E. coli. A temperature of the culture solution was kept at 30° C., and flask culture was performed until the OD₆₀₀ became 5 (approximately 15 hours) to obtain a seed culture solution.

TABLE 1
Seed culture medium (at the time of starting culturing, per 1 L)

| | | |
|---|---|---|
| Glucose | 5 | g |
| KH₂PO₄ | 4 | g |
| K₂HPO₄ | 10 | g |
| Yeast Extract | 6 | g |

Ampicillin was added, such that a final concentration became 100 mg/mL, to obtain a seed culture medium.

The seed culture solution was added to 500 ml of production medium (Table 2) such that the OD₆₀₀ became 0.05, to be inoculated with the transformed E. coli. The temperature of the culture solution was kept at 37° C., and culturing was performed by controlling a pH to be constant at 6.9. In addition, a dissolved oxygen concentration in the culture solution was maintained at 20% of a dissolved oxygen saturation concentration.

TABLE 2
Production, medium (at the time of starting culturing, per 1 L)

| | | |
|---|---|---|
| Glucose | 12 | g |
| KH₂PO₄ | 9 | g |
| MgSO₄•7H₂O | 2.4 | g |
| Yeast Extract | 15 | g |
| FeSO₄•7H₂O | 40 | mg |
| MnSO₄•5H₂O | 40 | mg |
| CeCl₂•2H₂O | 40 | mg |
| GD-113 (Antifoaming agent) | 0.1 | mL |

Immediately after glucose in the production medium was completely consumed, a feed solution (glucose 455 g/l L, Yeast Extract 120 g/l L) was added at a rate of 1 ml/min. The temperature of the culture solution was kept at 37° C., and culturing was perfomied by controlling a pH to be constant at 6.9. In addition, the dissolved oxygen concentration in the culture solution was maintained at 20% of the dissolved oxygen saturation concentration, and culturing was performed for 20 hours. Thereafter, 1 isopropyl-β-thiogalactopyranoside (IPTG) was added to the culture solution such that a final concentration became 1 mM, to induce expression of the target protein. At the time when 20 hours elapsed after the IPTG addition, the culture solution was centrifuged to recover fungus bodies. The SDS-PAGE was performed using the fungus bodies prepared from the culture solution before the IPTG addition and after the IPTG addition, and expression of target protein was confirmed by appearance of a band of a target protein size depending on the IPTG addition.

(3) Purification of Protein

Fungus bodies recovered after 2 hours from the IPTG addition were washed with 20 mM Tris-HCl buffer (pH 7.4). The fungus bodies after washed were suspended in 20 mM Tris-HCl buffer solution (pH 7.4) containing approximately 1 mM PMSF, and the cells were disrupted with a high-pressure homogenizer (GEANiro Soavi). The disrupted cells were centrifuged to obtain a precipitate. The resulting precipitate was washed with 20 mM Tris-HCl buffer solution (pH 7.4) to reach high purity. The precipitate after washed was suspended in 8 M guanidine buffer solution (8 M guanidine hydrochloride, 10 mM sodium dihydrogen phosphate, 20 mM NaCl, 1 mM Tris-HCl, and pH 7.0) such that a concentration became 100 mg/mL, and was stirred at 60° C. for 30 minutes with a stirrer to be dissolved. After dissolution, dialysis was performed with water by using a dialysis tube (cellulose tube 36/32 manufactured by Sanko Junyaku Co., Ltd.). White aggregated protein obtained after the dialysis was recovered by centrifugation, water was removed by a lyophilizes, and the lyophilized powder was recovered.

A degree of purification of the target protein in the obtained lyophilized powder was confirmed by image analysis of a result of polyacrylamide gel electrophoresis of the powder by using Total lab (nonlinear dynamics ltd.). As a result, the degree of purification of the protein was approximately 85%.

[Recovery of Spider Silk Protein]

### Example 1

A knit obtained by inter-knitting (mixing and knitting) the spider silk protein obtained above (artificial spider silk fiber, hereinafter also referred to as “SSP”) and a wool fiber which is a material different from the spider silk protein (hereinafter, also referred to as “other material”) was cut into approximately 2 cm square and approximately 1.79 g of knit was prepared. The respective weights of the spider silk protein and the wool fiber were 0.41 g and 1.38 g, by calculation. The knit and 6.78 g (water 4.75 g, CLYNSOLVE P-7 2.03 g) of mixed solvent of water as a polar solvent and CLYNSOLVE P-7 (a mixed solvent containing 85.5±1.0% of ethanol, less than 5.0% of isopropanol, 9.6±0.5% of normal propanol, and 0.2% or less of water, “CLYNSOLVE” is a registered trademark of Japan Alcohol Trading CO., LTD) (Water:CLYNSOLVE P-7=7:3) were input to a container with a stirrer and the container was sealed.

The container was set to a hot stirrer (manufactured by TOKYO RIKAKIKAI CO., LTD., RCH-20L), a heater temperature was set to 90° C., and stirring was performed at 300 rpm. The contents reached approximately 90° C. in first 10 minutes, and at the same time, internal pressure reached 0.08 MPa, by calculation, due to the vapor pressure of the solvent. From this state, stirring was further continued for 20 minutes. Thereafter, the container was removed from the hot stirrer and allowed to stand at a room temperature until the temperature fell to 70° C., which is equal to or lower than a boiling point of the solvent.

A lid of the container was detached, the stirrer was removed. Then, contents of the container were separated into a solution and insoluble matter by using a filter. Further, the insoluble matter was washed with water in the container, separated into a solution and insoluble matter by using a similar filter. This operation was repeated three times.

All filtered solutions were transferred to the same container and separated from the insoluble matter. The resulting solution and the insoluble matter were dried at 80° C. for 12 hours using a thermostat (PVH-212M manufactured by ESPEC Corp.), respectively.

After drying, respective weights of the solid matters obtained from the solution and the insoluble matter was measured. Results of respective weights of the spider silk protein and the wool fiber in the inter-knitted knit (before the treatment), respective weights thereof after the treatment, and yields thereof are shown in Table 3.

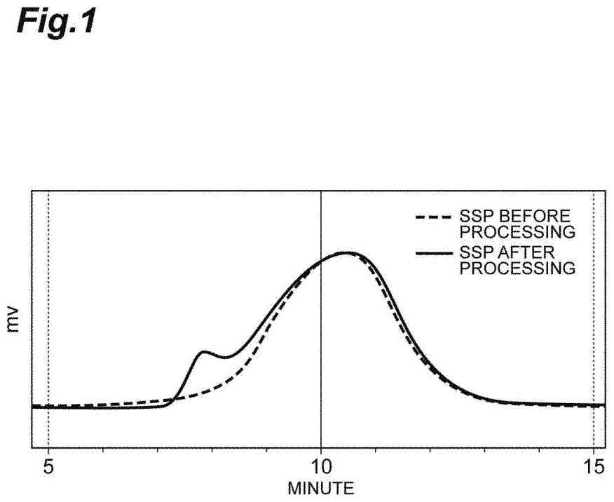

In addition, FIG. 1 shows results of GPC measurement of the spider silk protein before and after the treatment. As a result, since the results of the GPC measurement of the spider silk protein before and after the treatment were almost the same as each other, it was determined that the spider silk protein could be recovered almost as it was. In a graph after the treatment, presence of a substance is observed on a high molecular weight side (left side of the graph in FIG. 1), but it is considered that because the spider silk protein is aggregated by ethanol.

For the GPC measurement, GPC (manufactured by Shimadzu Corporation, trade name: 2C-20AD), column LF-404 (manufactured by Showa Denko), Shodex detector (manufactured by Showa Denko, trade name: RI-504) were used.

### Example 2

The wool fiber in Example 1 was changed to a cotton fiber, and the same experiment as in Example 1 was performed. Results thereof are shown in Table 3.

TABLE 3
Table 3. Polar solvent: Mixed solvent of water and alcohol

| | | | Used | | Weight | |
|---|---|---|---|---|---|---|
| | | | weight/g | | after | |
| | Another | Used | (Calculated | | treat- | |
| | material | weight/g | value) | | ment/g | Yield/% |
| Example 1 | Wool | 1.79 | SSP | 0.41 | 0.15 | 37.9% |
| | | | Wool | 1.38 | 1.58 | 114.5% |
| Example 2 | Cotton | 1.79 | SSP | 0.43 | 0.22 | 51.9% |
| | | | Cotton | 1.35 | 1.48 | 109.4% |

As described in Table 3, it was shown that, in a case where the mixed solvent of water and alcohol was used as the polar solvent, the SSP was recovered by applying pressure while heating the polar solvent containing the mixture of the SSP and another material. In addition, although the weight of the other material after the treatment is larger than that before processing, it is considered that a cause is that a part of the spider silk protein remains as attached.

### Example 3

The polar solvent in Example 1 was changed to water and the set temperature of the hot stirrer was changed to 110° C., and the same experiment as in Example 1 was performed. The contents reached approximately 110° C. in approximately 10 minutes from the start of the stirring, and at the same time, the internal pressure reached 0.15 MPa, by calculation, due to the vapor pressure of the solvent. In addition, the container was removed from the hot stirrer after the stirring, and thereafter, it was allowed to stand at a room temperature until the temperature of the container fell to 90° C., which is equal to or lower than a boiling point of the solvent. Results of respective weights of the spider silk protein and the cotton fiber in the inter-knitted knit (before the treatment), respective weights thereof after the treatment, and yields thereof are shown in Table 4.

### Example 4

The wool in Example 3 was changed to cotton, and the same experiment as in Example 3 was performed. Results thereof are shown in Table 4.

TABLE 4
Table 4. Polar solvent: Water

| | | | Used | | Weight | |
|---|---|---|---|---|---|---|
| | | | weight/g | | after | |
| | Another | Used | (Calculated | | treat- | |
| | material | weight/g | value) | | ment/g | Yield/% |
| Example 3 | Wool | 1.79 | SSP | 0.41 | 0.09 | 21.2% |
| | | | Wool | 1.38 | 1.67 | 120.8% |
| Example 4 | Cotton | 1.80 | SSP | 0.43 | 0.27 | 63.0% |
| | | | Cotton | 1.36 | 1.46 | 107.5% |

As described in Table 4, even in a case where water was used as the polar solvent, it was shown that the SSP was recovered from the mixture including the SSP and another material (wool or cotton).

Hereinafter, a similar experiment using a spider silk protein powder and a woven fabric of another fiber will be briefly described, instead of the knit obtained by inter-knitting the spider silk protein fiber and another fiber.

### Example 5

A plain-woven non-colored nylon fabric was cut into approximately 2 cm square and approximately 0.5 g of nylon fabric was prepared. This nylon fabric, 0.5 g of spider silk protein powder, and 4.5 g (water 3.15 g, CLYNSOLVE P-7 1.35 g) of a mixed solvent of water as a polar solvent and CLYNSOLVE (registered trademark) P-7 (Water:CLYNSOLVE P-7=7:3) were input to a container with a stirrer and the container was sealed.

The container was set to a hot stirrer (manufactured by TOKYO RIKAKIKAI CO., LTD., RCH-20L), a heater temperature was set to 90° C., and stirring was performed at 300 rpm. The contents reached approximately 90° C. in first 10 minutes, and at the same time, internal pressure reached 0.08 MPa, by calculation, due to the vapor pressure of the solvent. From this state, stirring was further continued for 20 minutes. Thereafter, the container was removed from the hot stirrer and allowed to stand at a room temperature until the temperature fell to 70° C., which is equal to or lower than a boiling point of the solvent.

A lid of the container was detached, the stirrer was removed. Then, contents of the container were separated into a solution and insoluble matter by using a filter. Further, the insoluble matter was washed with water in the container, separated into a solution and insoluble matter by using a similar filter. This operation was repeated three times.

All filtered solutions were transferred to the same container and separated from the insoluble matter. The resulting solution and the insoluble matter were dried at 80° C. for 12 hours using a thermostat (PVH-212M manufactured by ESPEC Corp.), respectively.

After drying, respective weights of the solid matters obtained from the solution and the insoluble matter was measured. Results of respective weights of the spider silk protein and the mixture (before the treatment) of the nylon fabric, respective weights thereof after the treatment, and yields thereof are shown in Table 5.

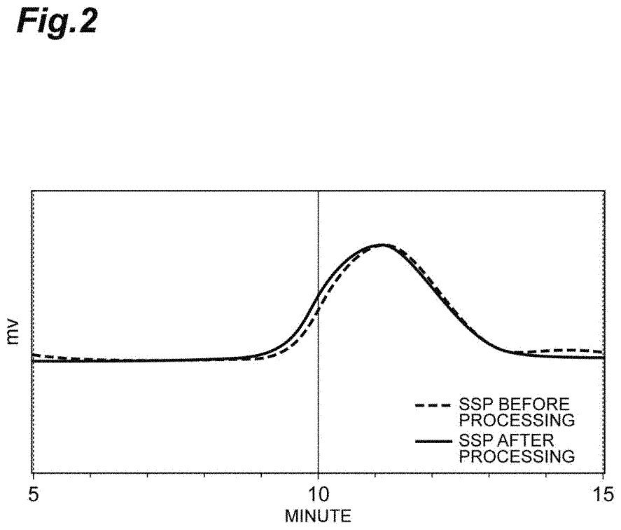

In addition, FIG. 2 shows results of GPC measurement of the spider silk protein before and after the treatment. As a result, since the results of the GPC measurement of the spider silk protein before and after the treatment were almost the same as each other, it was determined that the spider silk protein could be recovered almost as it was. The GPC measurement was carried out under the same conditions as in Example 1.

### Example 6

The non-colored nylon fabric in Example 5 was changed to a nylon fabric dyed in red, and the same experiment as in Example 5 was performed. Results thereof are shown in Table 5.

### Example 7

The non-colored nylon fabric in Example 5 was changed to a PET fabric dyed in blue, and the same experiment as in Example 5 was performed. Results thereof are shown in Table 5.

### Example 8

The non-colored nylon fabric in Example 5 was changed to a non-colored PET fabric, and the same experiment as in Example 5 was performed. Results thereof are shown in Table 5.

TABLE 5
Table 5. Polar solvent: Mixed solvent of water and alcohol

| | | | Used | | | |
|---|---|---|---|---|---|---|
| | | | weight/g | | Weight | |
| | | | (Calculated | | after | |
| | Another material | | value) | | treatment/g | Yield/% |
| Example 5 | Nylon | Non- | SSP | 0.50 | 0.49 | 96.9% |
| | | colored | Nylon | 0.51 | 0.50 | 99.3% |
| Example 6 | Nylon | Red | SSP | 0.50 | 0.40 | 79.9% |
| | | | Nylon | 0.51 | 0.54 | 105.9% |
| Example 7 | PET | Blue | SSP | 0.51 | 0.47 | 93.7% |
| | | | PET | 0.51 | 0.55 | 107.8% |
| Example 8 | PET | Non- | SSP | 0.51 | 0.49 | 95.9% |
| | | colored | PET | 0.50 | 0.52 | 103.3% |

As described in Table 5, it was shown that, in a case where the mixed solvent of water and alcohol was used as the polar solvent, even when the other material was any of the nylon and the PET, the spider silk protein can be recovered by applying pressure while heating the polar solvent containing the mixture of the SSP and another material. In addition, it was also shown that, even in a case of using another material dyed in red or blue, the spider silk protein was recovered.

### Example 9

The solvent in Example 5 was changed to water and the set temperature of the hot stirrer was changed to 110° C., and the same experiment as in Example 5 was performed. The contents reached approximately 110° C. in approximately 10 minutes from the start of the stirring, and at the same time, the internal pressure reached 0.15 MPa, by calculation, due to the vapor pressure of the solvent. In addition, the container was removed from the hot stirrer after the stirring, and thereafter, it was allowed to stand at a room temperature until the temperature of the container fell to 90° C., which is equal to or lower than a boiling point of the solvent. Results of respective weights of the spider silk protein and the mixture of the nylon fabric, respective weights thereof after the treatment, and yields thereof are shown in Table 6.

### Example 10

The non-colored nylon fabric in Example 9 was changed to a nylon fabric dyed in red, and the same experiment as in Example 9 was performed. Results thereof are shown in Table 6.

### Example 11

The non-colored nylon fabric in Example 9 was changed to a PET fabric dyed in blue, and the same experiment as in Example 9 was performed. Results are shown in Table 6.

### Example 12

The non-colored nylon fabric in Example 9 was changed to a non-colored PET fabric, and the same experiment as in Example 9 was performed. Results thereof are shown in Table 6.

TABLE 6
Table 4. Polar solvent: Water

| | | | Used | | | |
|---|---|---|---|---|---|---|
| | | | weight/g | | Weight | |
| | | | (Calculated | | after | |
| | Another material | | value) | | treatment/g | Yield/% |
| Example 9 | Nylon | Non- | SSP | 0.50 | 0.47 | 94.7% |
| | | colored | Nylon | 0.51 | 0.52 | 101.0% |
| Example 10 | Nylon | Red | SSP | 0.50 | 0.41 | 81.2% |
| | | | Nylon | 0.50 | 0.59 | 116.9% |
| Example 11 | PET | Blue | SSP | 0.51 | 0.27 | 52.7% |
| | | | PET | 0.51 | 0.60 | 118.0% |
| Example 12 | PET | Non- | SSP | 0.51 | 0.29 | 57.5% |
| | | colored | PET | 0.51 | 0.62 | 121.7% |

As described in Table 6, it was shown that, even in a case of using water as the polar solvent and nylon or PET as another material, the spider silk protein can be recovered. In addition, it was also shown that, even in a case of using another material dyed in red or blue, the spider silk protein was recovered.

1. A method for recovering protein, which recovers a target protein from a mixture containing the target protein and a material different from the target protein, the method comprising:
a dissolution step of dissolving either the target protein or the material by applying pressure while heating to a solution for dissolution containing the mixture and a polar solvent; and
a separation step of separating an obtained solution.

2. The method for recovering protein according to claim 1, wherein the dissolution step is a step of dissolving the target protein.

3. The method for recovering protein according to claim 1, wherein the target protein is one or more proteins selected from the group consisting of silk fibroin, spider silk fibroin, and hornet silk fibroin.

4. The method for recovering protein according to claim 1, wherein the polar solvent includes one or more solvents selected from the group consisting of water, alcohol, dimethyl sulfoxide, dimethylformamide, and hexafluoroacetone.

5. The method for recovering protein according to claim 1, wherein the material includes one or more materials selected from the group consisting of polyester, nylon, cotton, and wool.
